(19) United States
(12) Patent Application Publication
(10) Pub. No.: US 2023/0149444 A1
(43) Pub. Date: May 18, 2023

(54) HYPOCHLOROUS ACID SOLUTIONS AND METHODS OF USE

(71) Applicant: Phase One Health, LLC, Nashville, TN (US)

(72) Inventor: Robert Stephen Porter, Columbia, TN (US)

(21) Appl. No.: 17/916,591

(22) PCT Filed: Apr. 5, 2021
(86) PCT No.: PCT/US2021/025808

§ 371 Date: Oct. 3, 2022

Related U.S. Application Data
(60) Provisional application No. 63/005,174, filed on Apr. 3, 2020.

(51) Int. Cl. A61K 33/00; A61K 9/08; A01N 59/00; A61L 2/18; A61P 31/14; A61K 9/00

(52) U.S. Cl. CPC A61K 33/00; A61K 9/08; A01N 59/00; A61L 2/18; A61P 31/14; A61K 9/0078; A61L 2101/06

(57) ABSTRACT

Solutions comprising hypochlorous acid and methods of use are disclosed herein. The disclosed solutions are antiviral and antibacterial. The disclosed solutions may be used to treat and/or prevent a disease state or condition associated with or characterized by acute viral respiratory diseases, such as those caused by coronaviruses. The solutions may be administered by a nebulizer or a metered dose inhaler. The disclosed solutions may also be used to decontaminate surfaces. For example, the disclosed solutions may be used to decontaminate the surfaces of personal protective equipment, such as N95 face mask.

### CROSS-REFERENCE TO RELATED APPLICATIONS

This application cites the priority of U.S. 63/005,174, filed 3 Apr. 2020, and currently pending PCT/US2021/25808, filed 5 Apr. 2021. This application incorporates U.S. 62/991,554; U.S. 62/993,612; U.S. 63/005,174; and PCT/US2021/25808 herein by reference in their entireties.

### TECHNICAL FIELD

The present disclosure pertains to solutions comprising hypochlorous acid and methods of using said solutions for treatment of respiratory diseases and decontamination.

### TECHNICAL BACKGROUND

The present disclosure relates generally to methods of treatment and/or prevention of viral respiratory diseases and, specifically, of cold and flu-like diseases. These diseases include, but are not limited to, the common cold; seasonal flu; diseases cause by coronaviruses, including the novel coronavirus (i.e., COVID-19); sudden acute respiratory syndrome (SARS) and related diseases; Middle East Respiratory Syndrome (MERS); diseases caused by flu-like viruses, such as avian influenza virus (AIV), H7N1, H1N1, H3N2v, H1N5, etc.; and diseases that cause or compromise pulmonary function and/or secondary respiratory inflammation parenchymal damage, which often lead to acute respiratory distress syndrome (ARDS) and are sometimes referred to as viral influenza cytokine storm reactions. The cytokines associated with these viral maladies include interferon-alpha (IFN-α), interleukin-6 chemokine (C-C) ligand (CCL)2, and CCL5, and interferon-gamma (IFN-γ). These diseases are collectively referred to throughout the instant disclose as “acute viral respiratory diseases.”

Acute viral respiratory diseases can cause serious medical conditions and/or death. However, effective treatments are not readily accessible to people in need of the same, particularly outside of a hospital setting. Therefore, there is a need for readily-available methods of treatment for acute viral respiratory diseases that are effective, easily used by affected people, and are storable for a prolonged time period.

### DETAILED DESCRIPTION

The problems described above, as well as others, are addressed by the following inventions, although it is to be understood that not every embodiment of the inventions described herein will address each of the problems described above.

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terminology used herein is for the purpose of describing particular embodiments only and is not intended to be limiting. As used herein, the singular forms “a,” “an,” and “the” are intended to include the plural forms as well, unless the context dearly indicates otherwise.

Unless otherwise indicated, all numbers expressing quantities of solutions, percentages, temperatures, times, and so forth, as used in the specification or claims are to be understood as being modified by the term “about” or “approximately.” The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, terms such as “administering” or “administration” include acts such as prescribing, dispensing, giving, or taking a substance such that what is prescribed, dispensed, given, or taken actually contacts the patient's body externally or internally (or both). In embodiments of this disclosure, terms such as “administering” or “administration” include self-administering, self-administration, and the like, of a substance. Indeed, it is specifically contemplated that instructions or a prescription by a medical professional to a subject or patient to take or otherwise self-administer a substance is an act of administration.

As used herein, “disease” means any condition or disorder that damages or interferes with the normal function of a cell, tissue, or organ, including bone.

The terms “prevention,” “prevent,” “preventing,” “suppression,” “suppress,” and “suppressing”, as used herein, refer to a course of action (such as implanting a medical device) initiated prior to the onset of a clinical manifestation of a disease state or condition so as to prevent or reduce such clinical manifestation of the disease state or condition. Such preventing and suppressing need not be absolute to be useful.

The terms “treatment,” “treat,” and “treating” refer to a course of action (such as administering a solution) initiated after the onset of a clinical manifestation of a disease state or condition so as to eliminate or reduce such clinical manifestation of the disease state or condition. Such treating need not be absolute to be useful.

The term “in need of treatment,” as used herein, refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or device of the present disclosure.

The term “in need of prevention,” as used herein, refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method or device of the disclosure.

The terms “individual,” “subject,” or “patient,” as used herein, refer to any animal, including mammals, such as mice, rats, other rodents, rabbits, dogs, cats, swine, cattle, sheep, horses, or primates, and humans. The terms may specify male, female or both, or exclude male or female.

The term “therapeutically effective amount,” as used herein, refers to an amount of a compound, either alone or as a part of a pharmaceutical composition, that is capable of having any detectable, positive effect on any symptom, aspect, or characteristics of a disease state or condition. Such an effect need not be absolute to be beneficial.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

### B. Hypochlorous Acid Solutions

Acute viral respiratory diseases may be treated using hypochlorous acid (HOCl). Produced naturally by white blood cells (specifically, neutrophils), hypochlorous acid is an essential component of the human innate immune system, enabling the immune system to kill or destroy foreign pathogens, such as viruses, in the human body. Through a process known as respiratory burst, or oxidative burst, neutrophils release anti-bacterial, anti-fungal and virucidal chemicals, including hypochlorous acid.

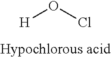

Once released, hypochlorous acid is a powerful oxidant that is able to rapidly inactivate bacteria and fungi, bacterial toxins, and viruses and degrade the membranes and proteins of bacteria and other internalized material. Due to its small size and neutral charge, HOCl is able to penetrate bacterial biofilm and spores. Additionally, HOCl presents anti-inflammatory activity by neutralizing inflammatory mediators, such as cytokines, in the body, including those exuded from pathogens.

Thus, hypochlorous acid is a critical component of the human body's defense mechanism against foreign pathogens, including viruses. However, humans have a limited capacity to produce hypochlorous acid through their immune systems. Moreover, humans having an immune system that has a diminished capacity (such as the elderly, immunocompromised or immunosuppressed) may not naturally produce sufficient HOCl to defend their bodies against foreign pathogens.

The present disclosure provides for a solution comprising hypochlorous acid. In some embodiments, the solution comprises pure hypochlorous acid. As used herein, the term “pure hypochlorous acid” generally refers to a solution of hypochlorous acid that is substantially free of impurities. Such impurities may, include, but are not limited to, sodium hypochlorite (NaClO), sodium perchlorate (NaClO4), plasticizers or other contaminants that may result from storage in plastic containers, and any compound that may increase the toxicity and/or decrease the virucidal effect of hypochlorous acid. In said embodiments, the pure hypochlorous acid may comprise less than 10%, less than 9%, less than 8%, less than 7%, less than 6%, less than 5%, less than 4%, less than 3%, less than 2%, less than 1%, less than 0.5%, less than 0.05%, less than 0.025%, less than 0.01%, less than 0.005%, or less than 0.0005% of impurities.

Pure hypochlorous acid solution may be prepared by various chemical processes known in the art,. in some embodiments, the pure hypochlorous acid solution may be prepared according to any of the methods for making an acidic solution disclosed in U.S. Pat. No. 7,393,522 and U.S. patent application Ser. No. 12/053,536, which are incorporated by reference herein. For example, the pure hypochlorous acid may be prepared by acidifying a NaClO solution with HCl. A non-limiting example of a process for preparing a hypochlorous acid solution is as follows:

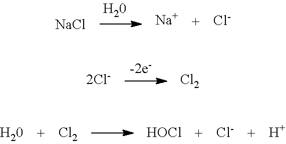

In some embodiments, the pure hypochlorous acid is substantially free of sodium hypochlorite (Na+—OCI), which is a primary ingredient in bleach. In said embodiments, the pure hypochlorous acid may comprise less than 10%, less than 9%, less than 8%, less than 7%, less than 6%, less than 5%, less than 4%, less than 3%, less than 2%, less than 1%, less than 0.5%, less than 0.05%, less than 0.025%, less than 0.01%, less than 0.005%, or less than 0.0005% of sodium hypochlorite.

Many HOCl-based solutions or products contain sodium hypochlorite. However, sodium hypochlorite is generally harmful (cytotoxic) when administered (e.g., ingested or inhaled) to subjects, such as humans. Furthermore, the presence of sodium hypochlorite may increase the toxicity of the HOCl-based solution or product as well as weaken its virucidal effect, as hypochlorite is a charged compound and, therefore, may have reduced injurious effect on foreign pathogens.

The solution may be aqueous. The term aqueous refers to a solution comprised of pure hypochlorous acid and a liquid, such as water or saline. In embodiments wherein the liquid is water, the water may be any suitable water. For example, the water may be medically or pharmaceutically acceptable water, including purified water and HPLC grade water. In embodiments wherein the solution is aqueous, the solution may have a hypochlorous acid concentration range of about 0.1 parts per million (ppm) to about 1000 ppm, a range of about 0.5 ppm to 750 ppm, and a range of about 5 ppm to 600 ppm, a range of about 1 ppm to 300 ppm, a range of about 60 ppm to 270 ppm, or any subrange or subvalue thereof. Some embodiments of the solution have a hypochlorous acid concentration of 250 ppm.

The solution of the disclosure may have a low pH. For example, the pH of the solution may range from about 2 to 6, from about 2.2 to 5.5, or from about 2.4 to 5. A specific embodiment of the solution has a pH of 5. The solution may have an oxidation-reduction potential (ORP) from about +100 millivolts (mV) to about +1200 mV, from about +600 mV to about +1200 mV, or from about +800 mV to +1190 mV, or from about +1000 mV to +1180 mV, or any subrange therebetween.

Because hypochlorous acid is a strong oxidant that is known to react with many different synthetic materials, the solution may be stored in a sealed container that tends not to react with oxidizing agents in order to maintain solution stability. As used herein, the term stable generally refers to the ability of the solution to maintain a portion of the HOC concentration for a specified period of time after preparation of the solution.

For example, the solution may be stored in a glass container. The glass container may be comprised of amber glass or coated soda lime amber glass. The glass container may have light protection, such as glass that is partially or fully opaque or a light-shielding film applied to the glass. Storage of the solution in amber glass has shown to preserve the long-term strength (potency) of the solution, namely, that the stored solution had a minimal reduction in ORP of HOCl as compared to the ORP of freshly prepared HOCl solution. For instance, it has been found that a solution of hypochlorous acid at 250 ppm (0.025%) stored under room temperature in amber glass is shelf stable for a significant period of time (for up to three years), which is long enough to be designated for home use and storage by patients affected by acute viral respiratory disease (e.g., prepared for use within two weeks).

The solution of the disclosure may have minimal to no cytotoxicity. For example, no substantial cytotoxicity was observed when a solution of this disclosure was applied in in vitro studies to the L929 cell line (fibroblasts derived from normal subcutaneous areolar adipose tissue) or when the solution was applied in in vivo studies to rabbit eyes. Indeed, in the aforementioned studies, solutions having an HOCl concentration of about 500 ppm or less resulted in no observable cytotoxicity. The solution of the present disclosure is believed to be nontoxic to biological tissues and comparable to sterile saline solutions. Indeed, because the solution is nontoxic and has virucidal properties, it is useful in any application in which virucidal properties are desirable, such as the treatment of viral infections.

### C. Methods of Treatment and Prevention

The teachings of the present disclosure provide a method of treatment and/or prevention of a disease or medical condition associated with or characterized by acute viral respiratory disease in a subject in need thereof, the method comprising administering any of the solutions disclosed above to the subject in a therapeutically effective amount. Acute viral respiratory diseases may be caused by one or more infectious viruses.

For example, the infectious virus may be an influenza virus. Influenza viruses belong to a family of viruses known as Orthomyxoviridae, and contain genetic information in the form of single-stranded RN A. The influenza virus may be any of the four types of influenza virus: types A, B, C, and D. Humans may be infected by types A, B, and C, whereas type D primarily infects livestock, such as cattle. Both influenza A and B viruses cause seasonal epidemics of disease on an annual basis (known as flu season). However, type A has the greater potential of causing widespread illness and global epidemics of flu disease. Other examples of infectious viruses include, but are not limited to, adenoviruses, human immunodeficiency virus (HIV), rhinoviruses, hepatitis (e.g., hepatitis A), coronavirus (responsible for Severe Acute Respiratory Syndrome Coronavirus (SARS-CoV)), including SARS-COVID-19, rotavirus, respiratory syncytial virus, herpes simplex virus, varicella-zoster virus, rubella virus, and other susceptible viruses.

The method of treatment and/or prevention comprises administering to the subject the solution in an amount sufficient to treat or prevent an acute viral respiratory disease. Any suitable method can be used for administering the solution to the subject. In one embodiment, the solution may be administered as a steam or a spray or by aerosolization, nebulization, or atomization. In such embodiments, the solution is inhaled by the subject.

In some embodiments, the solution is administered to a subject by nebulization. Medical nebulizers, for example, have been used to deliver a metered dose of a physiologically active liquid in an inspiration gas stream for inhalation by a recipient. Medical nebulizers can operate to generate liquid droplets, which form an aerosol with the inspiration gas. In embodiments wherein the solution is administered by nebulization, the solution may be administered in the form of droplets having a median size of no larger than 25 micrometers, 10 micrometers, 5 micrometers, 2 micrometers, or less than 2 micrometers, or any range or size therebetween. In some embodiments, the median droplet size of the solution ranges from about 2.5 micrometers to about 5 micrometers. Advantageously, a smaller median droplet size permits the solution to be deposited into the alveolar region of the lungs (the lower lung area), thus allowing for a greater therapeutic effect.

In some embodiments, the solution may be administered in the form of a single-use and/or metered-dose inhaler to provide a single dose or multiple doses. It is believed that administration by nebulization or by metered-dose inhaler is particularly effective for treating or preventing an acute viral respiratory disease, such as COVID-19, without deleterious effects in a subject. This is believed to be due to a number of factors, including for example, the small droplet size of the HOG solution that is produced and delivered to the respiratory system, minimal to no cytotoxicity, short duration of action, rapid virucidal killing and neutralization of cytokines, and anti-biofilm activity.

As detailed above, the hypochlorous acid of the solution is a strong oxidizing agent. Therefore, to maintain the stability of the solution, the nebulizer or inhaler may have a container for storing the solution that that tends not to react (e.g., is nonreactive) with oxidizing agents. For example, the nebulizer or inhaler may have a glass container (such as amber glass) for storing the solution.

The dosage of the administered solution may vary based on the subject in question (e.g., height, weight, gender, disease state or severity, etc. of the subject) Some embodiments of the method comprise administering about 1 milliliter (mL) to 10 mL of the solution per dose. Further embodiments of the method comprise administering about 3 mL to 7 mL of the solution per dose. A specific embodiment of the method comprises administering about 5 mL of the solution per dose. The solution may be administered to the subject in an amount per minute of at least 0.1 mL, 0.5 mL, 1 mL, 2 mL, 5 mL, 10 mL, 25 mL, 50 mL, 100 mL, 250 mL, 500 mL, or any range or subvalue thereof. The solution may be administered for periods of less than 1 minute, 1 minute, 5 minutes, 10 minutes, 15 minutes, or any duration of time therebetween. In a specific embodiment, the method comprises administering about 5 mL of a disclosed solution comprising 250 ppm HOCl to a subject via nebulization for about 5 to 10 minutes.

The solution may be administered one or more times. For example, the solution may be administered multiple times a day as needed (e.g., every hour, every 2 hours, every 4 hours, every 6 hours, every 12 hours, every morning and night, and so on) to treat acute viral respiratory diseases and/or symptoms related to acute viral respiratory disease.

In some embodiments, if, after administration of the solution, the subject still has acute viral respiratory diseases (e.g., tests positive for the respiratory disease in a viral diagnostic test), is at risk for acute viral respiratory diseases, or exhibits symptoms related to acute viral respiratory diseases, then the method may include one or more subsequent administrations of the solution. In some embodiments, the solution is administered for periods of time and at frequencies corresponding to the severity of symptoms of the acute viral respiratory disease. Symptoms related to acute viral respiratory disease may include, but are not limited to, fever, cough, pain, congestion, sore throat, fatigue, body ache, difficulty breathing, dizziness, low blood oxygen level, inflammation, such as inflammation of pulmonary tissue, cytokine storm response, evidence of pulmonary or respiratory compromise, and low arterial partial pressure of oxygen (PaO₂). For example, if, after administration of the solution, the subject continues to exhibit, for example, a cytokine storm response (e.g., a physician determines the subject has an overly active immune response through, for example, the detection of elevated serum ferritin values), then the method may include one or more subsequent administrations of the solution. The subsequent administrations may continue until the subject no longer exhibits a cytokine storm response (e.g., the subject no longer has elevated serum ferritin values).

A bioassay may also be conducted to determine the concentration of cytokines, such as IL-6 and IL-8, in conjunction with the administration of the solution. The bioassay may be conducted before the administration of the solution to determine a baseline cytokine concentration in, for example, affected tissue. Then, the solution may be administered. Then, another bioassay may be conducted to determine the new concentration of cytokines after the application of the solution. The subject may receive additional doses of the solution, administered in amounts correlated to the reduction in cytokine concentration identified in the bioassay. Administrations of the solution may cease when cytokines, such as IL-6 and IL-8, are present in low or undetectable levels in the affected tissue.

In some embodiments, the method includes diagnosing the subject as positive or presumptive positive for COVID-19 or negative for COVID-19. If the subject is diagnosed as positive or presumption positive for COVID-19, the method may include administering the solution. If the subject is not diagnosed as positive or presumption positive, the method may further include administering one or more of acetaminophen, nonsteroidal anti-inflammatory drugs, antihistamines, antitussives, steroids, decongestants, oxymetazoline, oseltamivir, or a pharmaceutically acceptable salt thereof in addition to, or instead of, administering the solution.

In some embodiments, the method may further include administering at least one compound to the subject in a therapeutically effective amount sufficient to treat or prevent a disease or medical condition associated with or characterized by acute viral respiratory disease. The at least one compound may be, for example, an anti-viral compound (e.g., β-D-N4-66 hydroxycytidine), an anti-malaria drug (e.g., hydroxychloroquine), an anti-bacterial drug (e.g., azithromycin), an anti-HIV drug (e.g., Biktarvy, Abacavir, Didanosine, Emtricitabine, Stavudine, Tenofovir alafenamide, Tenofovir disoproxil fumarate, and Zidovudine), an anti-flu drug (e.g., Favipiravir or Avigan, oseltamivir phosphate, zanamivir, peramivir, and baloxavir marboxil), an ACE inhibitor (e.g., benazepril hydrochloride, enalapril maleate, fosinopril sodium, lisinopril, moexipril, perindopril, quinapril hydrochloride, ramipril, and trandolapril), an ARS inhibitor (e.g., losartan, valsartan, irbesartan, candesartan, eprosartan mesylate, and telmisartan), a myeloperoxidase inhibitor (e.g., resveratrol and acetaminophen), and/or a vasodilator (e.g., nitric oxide). Any suitable method can be used for administering the compound to the subject. For example, the compound may be administered orally, by intravenous injection, by subcutaneous injection, or by inhalation, such as through nebulization. The dosage of the compound administered may vary based on the subject in question (for example, vary according to the weight, age, and/or gender of the subject) and the severity of symptoms of the acute viral respiratory disease of the subject. The compound may be administered before, after, or simultaneously with administration of the solution.

It is believed that the methods of treatment and/or prevention disclosed herein may provide one or more of the following benefits: rapid killing or destruction of viral bodies present in the subject, such as on the surface of the cells or tissue lining of the tracheobronchial tissues, lungs, or pulmonary tissue (e.g., in less than 10 seconds); the killing of secondary or related bacterial infections, such as but not limited to pneumococcal bacteria (e.g., Streptococcus pneumonia); the reduction or prevention of inflammation relating to cytokines, particularly IL6; reparative effects on the basement membrane and tissue; and reduction in the myeloperoxidase enzyme, the extended presence of which may cause other cardiovascular diseases.

### D. Methods of Decontamination

The disclosed solutions may be effective for decontamination. Disclosed herein are methods of decontaminating at least one surface of a target (e.g., an object and/or a material). The methods comprise contacting the surface of the target with any of the solutions disclosed herein to decontaminate the surface. As used herein, “decontamination” of a surface means killing, destroying, inactivating, and removing (collectively, referred to as “inactivating”) some or all of the infectious agent(s) at or on the surface. An “infectious agent” or “infectious agents” refers to any material or organism that causes disease in a host including, but not limited to, a virus, bacteria, prion, fungus, parasite, and disease (e.g., toxoplasmosis).

The decontamination may be partial (i.e., inactivation of some of the infectious agents on the target surface) or complete (i.e., inactivation of all of the infectious agents on the target surface). For example, the method may result in inactivation of 100%, 99%, 98%, 97%, 96%, 95%, 90%, 85.0%, 80%, or any subvalue thereof, of the infectious agents present on the surface. In a specific embodiment, the method comprises contacting a surface with a disclosed solution having an HOCl concentration of about 100-350 ppm, which results in inactivation of about 95% of the infectious agents on the surface.

The method may comprise contacting the target surface with a disclosed solution for any duration of time. The duration of time should be effective for partial or complete decontamination of the surface. For example, the duration of time may be less than 1 second, 2 seconds, 3 seconds, 4 seconds, 5 seconds, 10 seconds, 20 seconds, 30 seconds, 1 minute, 1.5 minutes, 2 minutes, 3 minutes, 4 minutes, 5 minutes, more than 5 minutes, or any subvalue therebetween.

In some embodiments, the method comprises removing the disclosed solution from the target surface. For example, the solution may be removed from the surface by wiping the solution off of the surface. In said embodiments, the solution may be wiped off with a clean and/or sterile wipe, cloth, rag, or the like. In some embodiments, the solution dries on the target.

The contacting step may be performed multiple times. A For example, a surface may be contacted with a disclosed solution more than once to decontaminate the surface. Multiple contacting steps may result in a higher degree of decontamination (i.e., more infectious agents are killed, destroyed, and/or inactivated upon each contacting step). In some embodiments, the surface may be contaminated with more infectious agents in-between contacting steps. That is, the surface may be decontaminated via the disclosed methods, exposed (or potentially exposed) to infectious agents, and then decontaminated again via the disclosed methods. This sequence may occur any number of times.

In some embodiments, the target may comprise personal protective equipment (PPE). In said embodiments, the method comprises contacting a disclosed solution with at least one surface of a PPE. Specifically, PPE is specialized clothing or equipment worn by a user (e.g., a healthcare worker) to decrease the likelihood of the user being exposed to infectious agents. For example, PPE may prevent a user from coming into contact with an infectious agent, or body fluid that may contain an infectious agent, by creating a barrier between the user and the infectious agent and/or body fluid. Examples of PPE include, but are not limited to, gloves, gowns, shoe covers, head covers, facial masks, respirators, eye protection, face shields, and goggles.

The PPE may be made of any material(s). In specific embodiments, the PPE comprises polyvinyl chloride (PVC) and/or silicone. Biocompatibility studies were performed on both of the aforementioned materials. These materials were contacted with solutions of varying HOCl concentrations for up to 24 hours. The biocompatibility studies demonstrated that the HOCl solutions were inert on both PVC and silicone.

Generally, PPE is discarded after a single use due to exposure (or potential exposure) to infectious agents. However, as described above, the disclosed solutions are antiviral and antibacterial. Thus, by contacting a disclosed solution with a PPE according to the aforementioned methods, the solution partially or completely decontaminates the PPE. Once decontaminated, the PPE may be re-worn by a user, with less risk of the user being exposed to an infectious agent. As a result, the PPE may be re-used, rather than discarded after a single use, which greatly reduces waste. Furthermore, in instances where PPE may be scarce, the disclosed methods provide a means of decontaminating limited PPE such that the PPE may be safely re-worn.

In a specific embodiment, the PPE is a respirator comprising a filter that filters airborne particles. For example, the respirator may comprise a facial mask, such as an N95 face mask. An N95 face mask is a mask that is designed to cover the mouth and nasal passages of a wearer and filters at least 95% of airborne particles. The N95 face mask uses materials (electret) with an electrostatic charge to clean air passing through the mask and remove the vast majority of contaminants in the air, such as infectious agents. Typical decontaminating agents, such as bleach, can destroy the elements of the N95 face mask that generate the electrostatic charge, which reduces or destroys the protective effect of the mark.

It is believed that contacting an N95 face mask with the disclosed solutions will not eliminate the mask's electrostatic charge. As a result, the N95 face mask will continue to generate a protective electrostatic charge, even after exposure to a solution. Advantageously, the disclosed solutions may not significantly reduce the performance (i.e., fit, appearance, filtration properties; etc.) of the N95 face mask. For example, after contacting the N95 face mask with a disclosed solution, the N95 face mask may be able to filter at least about 50%, at least about 60%, at least about 70%, at least about 80%, at least about 90%, or at least about 95% of airborne contaminants, including infectious agents.

In some embodiments, the disclosed solutions may not significantly, affect one or more of the following N95 face mask characteristics: a) changes in color or appearance, b) permeability or rejection performance, c) morphology, d) surface hydrophilicity, e) crystal structures or crystallinity, f) surface charge, and g) surface functional groups.

### E. Working Example

A male subject was diagnosed with COVID-19. The male was presenting with Headache, severe sore throat, Mile cough, low grade fever. On day 2 after diagnosis, the subject began self-administering an HOCl solution (HOCl concentration of 250 ppm in an aqueous solution). The solution was administered via nebulizer (droplet size of 2.5 to 5 μm) for 5 minutes every 2-4 hours.

After 5 days of self-administering the HOCl solution, the subject reported resolution of the sore throat no fever, and no headache. Throughout the duration of the subject's disease state, the subject never reported severe respiratory symptoms and subsequently tested negative for the virus.

### F. Conclusion

It is to be understood that any given elements of the disclosed embodiments of the disclosure may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses.

Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

I/We claim:

1. A method of treatment or prevention of a disease state or condition associated with or characterized by acute viral respiratory disease in a subject in need thereof, the method comprising administering a solution comprising hypochlorous acid to the subject in a therapeutically effective amount.

2. The method of claim 1, wherein the disease state or condition is caused by one or more virus.

3. The method of claim 2, wherein the one or more virus is a coronavirus.

4. The method of any one of claims 1-3, wherein the solution further comprises an aqueous component.

5. The method of claim 4, wherein the aqueous component is water.

6. The method of any one of claims 1-5, wherein the solution has a hypochlorous acid concentration of 0.025% of the solution.

7. The method of any one of claims 1-6, wherein the solution is administered by a nebulizer.

8. The method of any one of claims 1-6, wherein the solution is administered by a metered dose inhaler.

9. The method of claim 7 or 8, wherein the solution is administered in the form of droplets.

10. The method of claim 9, wherein the droplets have a median size of no larger than 5 micrometers.

11. The method of any one of claims 7-10, wherein the solution is inhaled by the subject.

12. The method of any one of claims 1-11, wherein the therapeutically effective amount of the solution is about 5 mL.

13. The method of any one of claims 1-12, wherein the solution is administered to the subject for a period between about 1 and 15 minutes.

14. The method of any one of claims 1-13, wherein the solution is administered between about 1 and 6 times per day.

15. The method of any one of claims 1-14, wherein the solution is substantially free of sodium hypochlorite.

16. The method of any one of claims 1-15, wherein the solution has a pH of about 2.4 to about 5.

17. The method of any one of claims 1-16, wherein the solution has a pH of about 5.

18. The method of any one of claims 1-17, wherein the solution has an oxidation reduction potential from about +800 to about +1190.

19. The method of any one of claims 1-18, wherein the solution has an oxidation reduction potential from about +100 to about +1180.

20. The method of any one of claims 1-19, wherein the solution is nontoxic to biological tissues.

21. The method of any one of claims 1-20, wherein the solution is stored in a container comprising amber glass.

22. The method of any one of claims 1-21, wherein administering the solution to a subject has an effect of at least one of: destroying virus bodies present in the subject; killing bacterial infections in the subject; reducing or preventing inflammation relating to cytokines in the subject; repairing basement membrane and tissue of the subject; reducing myeloperoxidase enzyme of the subject.

23. A solution for treating or preventing a disease state or condition associated with or characterized by acute viral respiratory disease, the solution comprising hypochlorous acid in a therapeutically effective amount.

24. The solution of claim 23, wherein the disease state or condition is caused by one or more virus.

25. The solution of claim 24, wherein the one or more virus is a coronavirus.

26. The solution of any one of claims 23-25, further comprising an aqueous component.

27. The solution of claim 26, wherein the aqueous component is water.

28. The solution of any one of claims 23-27, wherein the hypochlorous acid is present at a concentration of 0.025% of the solution.

29. The solution of any one of claims 23-28, wherein the solution is substantially free of sodium hypochlorite.

30. The solution of any one of claims 23-29, wherein the solution has a pH of about 2.4 to about 5.

31. The solution of any one of claims 23-30, wherein the solution has a pH of about 5.

32. The solution of any one of claims 23-31, wherein the solution has an oxidation reduction potential from about +800 to about +1190.

33. The solution of any one of claims 23-32, wherein the solution has an oxidation reduction potential from about +100 to about +1180.

34. The solution of any one of claims 23-33, wherein the solution is nontoxic to biological tissues.

35. The solution of any one of claims 23-34, wherein the solution is stored in a container comprising amber glass.

36. The solution of claim 1, wherein the solution is administered in combination with at least one of a group consisting of: an anti-viral compound, an anti-malaria drug, an anti-bacterial drug, an anti-HIV drug, an anti-flu drug, an ACE inhibitor, an ARB inhibitor, and a vasodilator.

37. A method of decontaminating a surface of a target comprising: contacting a surface of a target with a solution comprising hypochlorous acid.

38. The method of claim 37, wherein contacting the solution with the surface of the target has an effect of decontaminating the surface.

39. The method of claims 37 and 38, wherein the surface includes at least one infectious agent, and wherein contacting the solution with the surface has an effect of killing, destroying, removing, inactivating, or a combination thereof at least some of the at least one infectious agent.

40. The method of claim 39, wherein the at least infectious agent comprises a virus.

41. The method of claim 40, wherein the virus is a coronavirus.

42. The method of any one of claims 37-41, wherein the contacting occurs for a duration of time of less than 1 second, about 2 seconds, about 3 seconds, about 4 seconds, about 5 seconds, about 10 seconds, about 20 seconds, about 30 seconds, about 1 minute, about 1.5 minutes, about 2 minutes, about 3 minutes, about 4 minutes, about 5 minutes, or more than 5 minutes.

43. The method of any one of claims 37-42, wherein the method further comprises contacting the surface with the solution at least one additional time.

44. The method of any one of claims 37-43, wherein the method further comprises removing the solution from the surface.

45. The method of any one of claims 37-43, wherein the solution dries on the surface.

46. The method of any one of claims 37-45, wherein the target comprises personal protective equipment.

47. The method of claim 46, wherein the personal protective equipment comprises a respirator.

48. The method of claim 47, wherein the respirator comprises an N95 face mask.

49. The method of claim 48, wherein the N95 face mask filters at least 95% of airborne particles.

50. The method of claim 48 or 49, wherein the N95 face mask generates an electrostatic charge.

51. The method of claim 50, wherein contacting the surface of the N95 face mask with the solution does not eliminate the electrostatic charge of the N95 face mask.

52. The method of any one of claims 48-51, wherein contacting the surface of the N95 face mask with the solution does not significantly reduce performance of the N95 face mask.

53. The method of any one of claims 48-52, wherein contacting the surface of the N95 face mask with the solution does not significantly reduce filtration of the N95 face mask.

54. The method of any one of claims 48-53, wherein after contacting the surface of the N95 face mask with the solution, the N95 face mask filters at least 50%, at least 60%, at least 70%, at least 80%, at least 90%, or at least 95% of airborne particles.

55. The method of any one of claims 48-54, wherein contacting the surface of the N95 face mask with the solution does not significantly alter one or more of: color, appearance, permeability performance, rejection performance, morphology, surface hydrophilicity, crystal structures, crystallinity, surface charge, and surface functional groups of the N95 face mask.

56. The method of any one of claims 37-55, wherein the solution further comprises an aqueous component.

57. The method of claim 56, wherein the aqueous component is water.

58. The method of any one of claims 37-57, wherein the solution has a hypochlorous acid concentration of 0.025% of the solution.

59. The method of any one of claims 37-58, wherein the solution is substantially free of sodium hypochlorite.

60. The method of any one of claims 37-59, wherein the solution has a pH of about 2.4 to about 5.

61. The method of any one of claims 37-60, wherein the solution has a pH of about 5.

62. The method of any one of claims 37-61, wherein the solution has an oxidation reduction potential from about +800 to about +1190.

63. The method of any one of claims 37-62, wherein the solution has an oxidation reduction potential from about +100 to about +1180.

64. The method of any one of claims 37-63, wherein the solution is nontoxic to biological tissues.

65. The method of any one of claims 37-64, wherein the solution is stored in a container comprising amber glass.
